(19) United States
(12) Patent Application Publication
(10) Pub. No.: US 2026/0234081 A1
(43) Pub. Date: Aug. 13, 2026

(54) METHOD FOR PREPARING ETHYLENE FROM CHLORINATED ORGANIC WASTE

(71) Applicant: Zhejiang University, Hangzhou (CN)

(72) Inventors: Qiwei YANG, Hangzhou (CN); Yanping YU, Hangzhou (CN); Wenjun ZHANG, Hangzhou (CN); Qilong REN, Hangzhou (CN); Baogen SU, Hangzhou (CN); Zhiguo ZHANG, Hangzhou (CN); Zongbi BAO, Hangzhou (CN)

(21) Appl. No.: 19/640,656

(22) Filed: Apr. 7, 2026

Related U.S. Application Data
(63) Continuation of application No. PCT/CN2025/140024, filed on Dec. 4, 2025.

(30) Foreign Application Priority Data
Dec. 4, 2024 (CN) 202411764674.9

(51) Int. Cl. C07C 1/26; B01J 19/08; C07C 11/04

(52) U.S. Cl. CPC C07C 1/26; B01J 19/088; C07C 11/04; B01J 2219/00123; B01J 2219/0845; B01J 2219/0894

(57) ABSTRACT

A method for preparing ethylene from chlorinated organic waste is provided, which belongs to the technical field of chemical engineering and environmental protection. By using a gas containing hydrogen element and carbon element as a working gas, the chlorinated organic waste is treated and converted into high value-added chemicals such as ethylene and acetylene, thereby achieving resource utilization of chlorinated organic waste. The working gas not only provides a hydrogen-rich reaction atmosphere but also provides a carbon source. Meanwhile, a staged quenching technology is adopted.

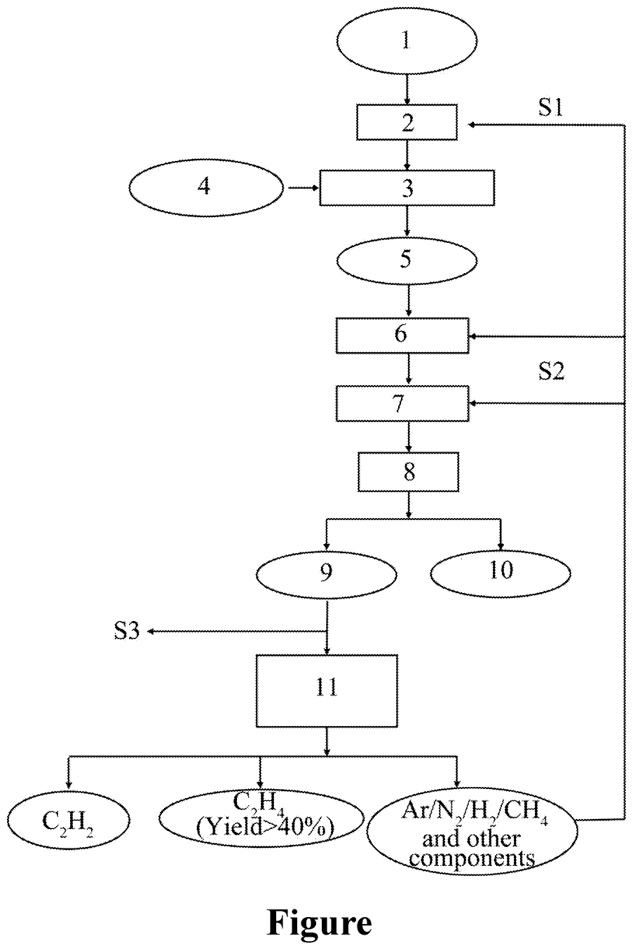

### CROSS REFERENCE TO RELATED APPLICATION

The present application is a continuation application of International Patent Application No. PCT/CN2025/140024, filed on Dec. 4, 2025, which claims priority to Chinese Patent Application No. CN202411764674.9, filed with the China National Intellectual Property Administration on Dec. 4, 2024 and entitled “Method for preparing ethylene from chlorinated organic waste”. The disclosure of the two applications is incorporated by references herein in their entireties as part of the present application.

### TECHNICAL FIELD

The present disclosure belongs to the technical field of chemical engineering and environmental protection, and in particular relates to a method for preparing ethylene from chlorinated organic waste.

### BACKGROUND

Currently, large quantities of chlorinated waste are generated in the chemical, pharmaceutical, pesticide, and other industries. The primary organic constituents of such waste include chlorinated alkanes, chlorinated alkenes, chlorinated benzene compounds, etc. Chlorinated organic waste is a category of hazardous waste that typically exhibits high toxicity, infectivity, and high residue accumulation. Due to the significant hazards it poses to the ecological environment and human health, treatment technologies are required to achieve an efficiency approaching 100%. Meanwhile, recycling chlorinated organic waste to prepare high value-added chemicals can conserve significant resources. This approach further achieves resource utilization on the basis of realizing waste reduction and harmless treatment.

Currently, conventional treatment methods for chlorinated organic waste include incineration and landfilling. Incineration is capable of substantially reducing the volume and mass of waste and rendering it harmless. However, on the one hand, the organic chlorine in chlorinated organic waste inhibits combustion, lowering combustion efficiency; furthermore, due to the low calorific value of such waste, supplementary fuel is required for co-incineration, which increases the cost of incineration. On the other hand, improper operation during incineration can easily lead to the generation of highly toxic dioxins, causing secondary pollution. Although landfill disposal is simple and easy to implement, the presence of chlorine increases the difficulty of waste disposal. In the case of direct landfilling, plasticizers and stabilizers may leach out, contaminating the soil and groundwater. Moreover, landfilling occupies a large amount of land. As land resources become increasingly scarce, the cost of safe landfilling also rises significantly.

Therefore, there is an urgent need for a technology capable of achieving both the harmless treatment and resource utilization of chlorinated organic waste.

### SUMMARY

An object of the present disclosure is to provide a method for preparing ethylene from chlorinated organic waste. The method provided by the present disclosure enables the harmless treatment and resource utilization of chlorinated organic waste.

To achieve the above object, the present disclosure provides the following technical solution.

The present disclosure provides a method for preparing ethylene from chlorinated organic waste, including the following steps:
forming a plasma atmosphere from a working gas, and subjecting the chlorinated organic waste to pyrolysis reaction and staged quenching in sequence in the plasma atmosphere to obtain a pyrolysis product, where the staged quenching includes a first quenching and a second quenching performed in sequence, a quenching rate of the first quenching being greater than a quenching rate of the second quenching; and the working gas contains hydrogen element and carbon element, a molar ratio of the hydrogen element to the carbon element in the plasma atmosphere being greater than 2:1; and
subjecting the pyrolysis product to gas-solid separation to obtain a gas-phase product and a solid-phase product, where the gas-phase product includes ethylene.

In some embodiments, the quenching rate of the first quenching is greater than or equal to 1×10⁴ K/s, and a product obtained after the first quenching has a temperature of 1100-1300 K.

In some embodiments, the quenching rate of the second quenching is in a range of 300-1000 K/s, and the pyrolysis product has a temperature of less than 600 K.

In some embodiments, the working gas includes at least one selected from the group consisting of dry gas, coke oven gas, and natural gas.

In some embodiments, the chlorinated organic waste includes carbon element and chlorine element.

In some embodiments, the pyrolysis reaction is carried out at a temperature of 1800-3500 K.

In some embodiments, the staged quenching is carried out by means including inter-wall quenching and/or direct quenching; the direct quenching includes physical quenching and/or chemical quenching; a cooling medium used in the inter-wall quenching includes one selected from the group consisting of water, liquid nitrogen, a cooling oil, and a coolant; and a cooling medium used in the direct quenching includes at least one selected from the group consisting of argon, nitrogen, propane, and the working gas.

In some embodiments, the pyrolysis reaction is carried out in a plasma reactor; and the plasma reactor includes one selected from the group consisting of a radio frequency plasma reactor, a microwave plasma reactor, and an arc plasma reactor.

In some embodiments, a flow rate of the working gas is in a range of 0.5-1500 Nm³/h; and the chlorinated organic waste is treated in an amount of 0.5-1000 kg/h.

In some embodiments, the chlorinated organic waste is introduced into the plasma reactor using a carrier gas, and the carrier gas includes at least one selected from the group consisting of dry gas, coke oven gas, hydrogen, and argon.

The present disclosure provides a method for preparing ethylene from chlorinated organic waste, including the following steps: forming a plasma atmosphere from a working gas, and subjecting the chlorinated organic waste to pyrolysis reaction and staged quenching in sequence in the plasma atmosphere to obtain a pyrolysis product, where the staged quenching includes a first quenching and a second quenching in sequence, a quenching rate of the first quenching being greater than a quenching rate of the second quenching; and the working gas contains hydrogen element and carbon element, a molar ratio of the hydrogen element to the carbon element in the plasma atmosphere being greater than 2:1; and subjecting the pyrolysis product to gas-solid separation to obtain a gas-phase product and a solid-phase product, where the gas-phase product includes ethylene.

The present disclosure employs an efficient and environmentally-friendly thermal plasma technology. By using a gas containing hydrogen element and carbon element as a working gas, the chlorinated organic waste is treated and converted into high value-added chemicals such as ethylene and acetylene, thereby achieving the resource utilization of chlorinated organic waste. The working gas not only provides a hydrogen-rich reaction atmosphere but also provides a carbon source, thereby increasing the production of ethylene and acetylene. Meanwhile, a staged quenching technology is adopted. By controlling the quenching rates within different temperature ranges during the quenching process, the selectivity and yield of ethylene are greatly improved, addressing the problem of low ethylene production in pyrolysis gas-phase products in the prior art and achieving effective regulation of the composition of the gas-phase products from the pyrolysis of chlorinated organic waste.

Further, the method according to the present disclosure exhibits good adaptability to feedstocks, imposes no special requirements on the state and composition of chlorinated organic waste, possesses good applicability, and requires no pretreatment. Furthermore, there are no special requirements on the type and specific composition of the working gas. The feedstock utilization is high; specifically, the conversion rate of chlorinated organic waste is greater than 98%, and the ethylene content in the pyrolysis gas is significantly increased. The equipment used is simple and easy to operate, no catalyst is required, and the method of the present disclosure is environmentally-friendly, generates no harmful substances such as dioxins, and achieves zero emissions of pollutants.

The method provided by the present disclosure has the advantages of short reaction time, high conversion rate, no secondary pollution, and controllable products. It achieves comprehensive resource utilization of chlorinated organic waste and significantly increases the production of ethylene in the products.

### BRIEF DESCRIPTION OF THE DRAWINGS

FIGURE shows a schematic flowchart of the method according to an embodiment of the present disclosure. In the FIGURE, 1 represents chlorinated organic waste; 2 represents feed system; 3 represents plasma reactor; 4 represents dry gas as working gas; 5 represents pyrolysis product; 6 represents high-temperature quenching zone; 7 represents low-temperature quenching zone; 8 represents gas-solid separation; 9 represents gas-phase product; 10 represents solid-phase product; and 11 represents conventional separation devices such as absorption and distillation; S1 represents recycle to feed system as carrier gas; S2 represents recycle to high-temperature/low-temperature quenching zones as quenching medium for reuse; and S3 represents vinyl chloride production section using alkene-alkyne process.

### DETAILED DESCRIPTION OF THE EMBODIMENTS

The present disclosure provides a method for preparing ethylene from chlorinated organic waste, including the following steps:
forming a plasma atmosphere from a working gas, and subjecting the chlorinated organic waste to pyrolysis reaction and staged quenching in sequence in the plasma atmosphere to obtain a pyrolysis product, where the staged quenching includes a first quenching and a second quenching performed in sequence, a quenching rate of the first quenching being greater than a quenching rate of the second quenching; and the working gas contains hydrogen element and carbon element, a molar ratio of the hydrogen element to the carbon element in the plasma atmosphere being greater than 2:1; and
subjecting the pyrolysis product to gas-solid separation to obtain a gas-phase product and a solid-phase product, where the gas-phase product includes ethylene.

The present disclosure involves forming a plasma atmosphere from a working gas, and subjecting the chlorinated organic waste to pyrolysis reaction and staged quenching in sequence in the plasma atmosphere to obtain a pyrolysis product.

In the present disclosure, the working gas contains hydrogen element and carbon elements. In the present disclosure, a molar ratio of the hydrogen element to the carbon element in the plasma atmosphere is greater than 2:1, further preferably 4-20:1, and specifically may be 4.9:1, 5.2:1, 5.6:1, 6.5:1. In some embodiments of the present disclosure, the working gas includes at least one selected from the group consisting of dry gas, coke oven gas, and natural gas. In some embodiments, the dry gas includes at least one selected from the group consisting of reforming dry gas, hydrocracking dry gas, catalytic cracking dry gas, and coking dry gas.

The process for forming the plasma atmosphere is not specifically limited in the present disclosure, and any process that is well known to the skilled in the art may be used.

In some embodiments of the present disclosure, the chlorinated organic waste contains carbon element and chlorine element, and may also contain hydrogen element. In the present disclosure, there is no special requirement on the source and state of the chlorinated organic waste, and ratio of the elements therein, and those well known to the skilled in the art may be used. In a specific embodiment of the present disclosure, the chlorinated organic waste is solid hexachlorobenzene waste or liquid tetrachloroethylene waste provided by a chemical plant.

In some embodiments of the present disclosure, the pyrolysis reaction is carried out in a plasma reactor. In some embodiments, the plasma reactor is selected from the group consisting of a radio frequency plasma reactor, a microwave plasma reactor, and an arc plasma reactor. In some embodiments, the arc plasma reactor is a direct-current arc plasma reactor. In some embodiments, the direct-current arc plasma reactor is a magnetically rotating arc thermal plasma reactor. In the present disclosure, a magnetically rotating arc thermal plasma reactor is used, in which the arc rotates at high speed, which is beneficial for extending the service life of the anode, and simultaneously enables more sufficient mixing between the feedstock and the plasma, thereby improving the pyrolysis reaction efficiency.

In some embodiments of the present disclosure, under a condition that the chlorinated organic waste is solid hexachlorobenzene waste, the chlorinated organic waste is introduced into the plasma reactor via a solid feed system. In some embodiments, under a condition that the chlorinated organic waste is liquid tetrachloroethylene waste, the chlorinated organic waste is pumped into the plasma reactor, preferably using a peristaltic pump.

In some embodiments of the present disclosure, a flow rate of the working gas is in a range of 0.5-1500 Nm³/h. In some embodiments of the present disclosure, the chlorinated organic waste is treated in an amount of 0.5-1000 kg/h. In some embodiments of the present disclosure, the chlorinated organic waste is introduced into the plasma reactor using a carrier gas. In some embodiments, the carrier gas includes at least one selected from the group consisting of dry gas, coke oven gas, hydrogen, and argon. In the present disclosure, the appropriate flow rates of the working gas and carrier gas are selected based on the feed amount of chlorinated organic waste and the required hydrogen/carbon element ratio. In the present disclosure, the flow rate of the carrier gas is not particularly limited, provided that it is sufficient to fluidize the chlorinated organic waste into the plasma reactor.

In some embodiments of the present disclosure, under a condition that an arc plasma reactor is used to treat the chlorinated organic waste, the chlorinated organic waste is introduced into the arc plasma reactor from up or bottom of the arc; and more preferably, the chlorinated organic waste is introduced into the arc plasma reactor from up of the arc. This allows for full utilization of the heat in the arc region, and facilitates the sufficient mixing of the feedstock with the thermal plasma, thereby ensuring that the pyrolysis reaction proceeds rapidly and sufficiently.

In some embodiments of the present disclosure, the pyrolysis reaction is carried out at a temperature of 1800-3500 K, which specifically may be 1800 K, 2000 K, 2200 K, 2400 K, 2500 K, 2800 K, 3000 K, 3200 K, or 3500 K.

In some embodiments of the present disclosure, a quenching rate of the first quenching is greater than or equal to 1×10⁴ K/s. In some embodiments, a product obtained after the first quenching has a temperature of 1100-1300 K. In some embodiments of the present disclosure, a quenching rate of the second quenching is in a range of 300-1000 K/s. In some embodiments, the pyrolysis product has a temperature of less than 600 K. In the present disclosure, the quenching rate is adjusted by changing the internal wall structure of the quenching section, the type of cooling medium, and/or the flow rate. The quenching rate has a significant influence on the composition of the pyrolysis gas. If the quenching rate in the high-temperature quenching section (i.e., the first quenching) is too low, acetylene would rapidly decompose into solid carbon and hydrogen gas at high temperature, greatly reducing the total yield of hydrocarbons in the gas-phase product. If the quenching rate in the low-temperature quenching section (i.e., the second quenching) is too high, the hydrogenation of acetylene into ethylene cannot proceed sufficiently, reducing the production of ethylene in the gas-phase product.

In some embodiments of the present disclosure, the staged quenching is carried out by means including inter-wall quenching and/or direct quenching. In some embodiments, the direct quenching includes physical quenching and/or chemical quenching. In some embodiments, a cooling medium used in the inter-wall quenching is selected from the group consisting of water, liquid nitrogen, a cooling oil, and a coolant. In some embodiments, a cooling medium used in the direct quenching includes at least one selected from the group consisting of argon, nitrogen, propane, and the working gas. In some embodiments of the present disclosure, the cooling medium is sprayed into the quenching zone of the plasma reactor in a radial or tangential direction.

In the present disclosure, after obtaining the pyrolysis product, the pyrolysis product is subjected to gas-solid separation to obtain a gas-phase product and a solid-phase product, where the gas-phase product includes ethylene.

In the present disclosure, there is no special requirement on the gas-solid separation method, and conventional gas-solid separation technology in the art may be used. In some embodiments, a cyclone separator, a bag filter, or a ceramic separator is used, and specifically, a ceramic separator may be used.

In the present disclosure, the gas-phase product includes ethylene, and preferably also includes acetylene and hydrogen chloride. In some embodiments of the present disclosure, after obtaining the gas-phase product, the method further includes conveying the gas-phase product to a vinyl chloride production section using an alkene-alkyne process, or subjecting the gas-phase product to conventional separation and purification operations such as absorption and distillation to obtain ethylene and acetylene products. In some embodiments, hydrogen, argon, and other gases obtained after separation can be returned to the plasma device to serve again as a carrier gas and/or a quenching medium to participate in the transport and pyrolysis of the chlorinated organic waste, thereby improving the gas utilization rate.

In some embodiments of the present disclosure, the solid-phase product is carbon. In some embodiments, the elemental carbon is present in the form of nanoscale carbon black particles and/or graphene.

In some embodiments of the present disclosure, the conversion rate of the chlorinated organic waste is greater than 98%. In some embodiments, the yield of ethylene in the pyrolysis gas is greater than 40%.

The schematic flowchart of the method provided by the present disclosure is shown in FIGURE, where the working gas is dry gas, the high-temperature quenching zone corresponds to the first quenching zone, and the low-temperature quenching zone corresponds to the second quenching zone.

Unless otherwise specified, the materials and equipment used in the present disclosure are commercially available in the art.

The technical solutions in the present disclosure will be described clearly and completely below in conjunction with the examples of the present disclosure. Apparently, the described examples are merely some rather than all of the examples of the present disclosure. Based on the examples of the present disclosure, all other examples that would have been obtained by those of ordinary skill in the art without any creative efforts shall fall within the scope of the present disclosure.

### Example 1

Chlorinated organic waste (solid hexachlorobenzene waste provided by a chemical plant) was fed into a microwave plasma reactor via a solid feed system using a carrier gas, where the feed rate of hexachlorobenzene was 2.8 kg/h, a working gas used was dry gas with a flow rate of 5 Nm³/h, the carrier gas was argon with a flow rate of 3.3 Nm³/h, the temperature in the plasma reactor was 2000 K, and the molar ratio value of the hydrogen element to the carbon element in the plasma atmosphere was 4.9. After a millisecond-level pyrolysis reaction, the resulting pyrolysis product was subjected to a first quenching and a second quenching in sequence until the temperature of the pyrolysis product was 600 K or lower, where the quenching rate of the first quenching was 1×10⁴ K/s, the temperature of a product obtained after the first quenching was 1200 K, the quenching rate of the second quenching was 500 K/s, and means for quenching was indirect heat transfer using deionized water as a cooling medium.

The pyrolysis product obtained after quenching was subjected to gas-solid separation using a ceramic separator to obtain a gas-phase product and a solid-phase product.

As determined by gas chromatography, main components of the gas-phase product (excluding the working gas and the carrier gas) were ethylene, acetylene, and hydrogen chloride, with volume fractions of 19.66%, 19.26%, and 48.13%, respectively. The yield of ethylene was 40.64%, and the yield of acetylene was 39.82%. Transmission electron microscopy-energy dispersive spectrometry (TEM-EDS) elemental analysis revealed that the solid product was mainly composed of C, O, and Cl elements, with C accounting for 96.7% and Cl accounting for 0.33%. The solid product was mainly nanoscale carbon black particles.

### Example 2

Chlorinated organic waste (solid hexachlorobenzene waste provided by a chemical plant) was fed into a radio frequency plasma reactor via a solid feed system using a carrier gas, where the feed rate of hexachlorobenzene was 10 kg/h, a working gas used was dry gas with a flow rate of 20 Nm³/h, the carrier gas was argon with a flow rate of 8 Nm³/h, the temperature in the plasma reactor was 2400 K, and the molar ratio value of the hydrogen element to the carbon element in the plasma atmosphere was 5.2. After a millisecond-level pyrolysis reaction, the resulting pyrolysis product was subjected to a first quenching and a second quenching in sequence until the temperature of the pyrolysis product was 600 K or lower, where the quenching rate of the first quenching was 1.2×10⁴ K/s, the temperature of a product obtained after the first quenching was 1100 K, the quenching rate of the second quenching was 500 K/s, and means for quenching was direct heat exchange, in which argon gas was sprayed radially into the plasma reactor as a cooling medium.

The pyrolysis product obtained after quenching was subjected to gas-solid separation using a ceramic separator to obtain a gas-phase product and a solid-phase product.

As determined by gas chromatography, main components of the gas-phase product (excluding the working gas and carrier gas) were ethylene, acetylene, and hydrogen chloride, with volume fractions of 20.59%, 18.16%, and 46.92%, respectively. The yield of ethylene was 42.11%, and the yield of acetylene was 37.15%. The solid-phase product was mainly a mixture of nanoscale carbon black particles and graphene.

### Example 3

Chlorinated organic waste (solid hexachlorobenzene waste provided by a chemical plant) was fed into a magnetically rotating arc thermal plasma reactor via a solid feed system using a carrier gas, where the feed rate of hexachlorobenzene was 100 kg/h, a working gas used was dry gas with a flow rate of 175 Nm³/h, the carrier gas was dry gas with a flow rate of 75 Nm³/h, the temperature in the plasma reactor was 3000 K, and the molar ratio value of the hydrogen element to the carbon element in the plasma atmosphere was 5.6. After a millisecond-level pyrolysis reaction, the resulting pyrolysis product was subjected to a first quenching and a second quenching in sequence until the temperature of the pyrolysis product was 600 K or lower, where the quenching rate of the first quenching was 1.5×10⁴ K/s, the temperature of a product obtained after the first quenching was 1200 K, the quenching rate of the second quenching was 400 K/s, and means for quenching was direct heat exchange, in which argon gas was sprayed radially into the plasma reactor as a cooling medium.

The obtained pyrolysis product was subjected to gas-solid separation using a ceramic separator to obtain a gas-phase product and a solid-phase product.

As determined by gas chromatography, main components of the gas-phase product (excluding the working gas and carrier gas) were ethylene, acetylene, and hydrogen chloride, with volume fractions of 21.81%, 14.43%, and 41.51%, respectively. The yield of ethylene was 43.05%, and the yield of acetylene was 28.49%. The solid-phase product was mainly a mixture of nanoscale carbon black particles and graphene.

### Example 4

Chlorinated organic waste (liquid tetrachloroethylene waste provided by a chemical plant) was fed into a magnetically rotating arc thermal plasma reactor via a peristaltic pump using a carrier gas, where the feed rate of tetrachloroethylene was 1000 kg/h, a working gas used was dry gas with a flow rate of 2000 Nm³/h, the carrier gas was argon with a flow rate of 550 Nm³/h, the temperature in the plasma reactor was 2500 K, and the molar ratio value of the hydrogen element to the carbon element in the plasma atmosphere was 6.5. After a millisecond-level pyrolysis reaction, the resulting pyrolysis product was subjected to a first quenching and a second quenching in sequence until the temperature of the pyrolysis product was 600 K or lower, where the quenching rate of the first quenching was 1.5×10⁴ K/s, the temperature of a product obtained after the first quenching was 1200 K, the quenching rate of the second quenching was 400 K/s, and means for quenching was direct heat exchange, in which argon gas was sprayed radially into the plasma reactor as a cooling medium.

The obtained pyrolysis product was subjected to gas-solid separation using a ceramic separator to obtain a gas-phase product and a solid-phase product.

As determined by gas chromatography analysis, main components of the gas-phase product (excluding the working gas and carrier gas) were ethylene, acetylene, and hydrogen chloride, with volume fractions of 15.92%, 15.84%, and 54.14%, respectively. The yield of ethylene was 41.24%, and the yield of acetylene was 41.01%. The flow rates of ethylene and acetylene in the gas-phase product were 158.75 Nm³/h and 157.88 Nm³/h, respectively. The solid-phase product was mainly a mixture of nanoscale carbon black particles and graphene.

### Comparative Example 1

Ethylene was prepared according to the method in Example 3, except that the quenching process was different. Specifically, the resulting pyrolysis product was quenched at a quenching rate of 1.5×10⁴ K/s until the temperature of the pyrolysis product was 600 K or lower, where means for quenching was direct heat exchange, in which argon gas was sprayed radially into the plasma reactor as a cooling medium.

As determined by gas chromatography, main components of the gas-phase product (excluding the working gas and carrier gas) were ethylene, acetylene, and hydrogen chloride, with volume fractions of 7.41%, 41.71%, and 47.08%, respectively. The yield of ethylene was 12.91%, and the yield of acetylene was 72.58%.

### Comparative Example 2

Ethylene was prepared according to the method of Example 4, except that hydrogen was used as the working gas instead of dry gas.

As determined by gas chromatography analysis, main components of the gas-phase product (excluding the working gas and carrier gas) were ethylene, acetylene, and hydrogen chloride, with volume fractions of 8.09%, 7.50%, and 78.16%, respectively. The yield of ethylene was 41.38%, and the yield of acetylene was 38.39%. However, the flow rates of ethylene and acetylene in the gas-phase product were 55.84 Nm³/h and 51.80 Nm³/h, respectively, which were significantly lower than those of ethylene and acetylene in Example 4. It demonstrates that the method provided by the present disclosure can further increase the production of ethylene and acetylene.

Although the embodiments described above have provided a detailed description of the present disclosure, they are only a part of, rather than all of the embodiments of the present disclosure. All other embodiments that can be obtained according to the embodiments of the present disclosure without creative efforts shall fall within the scope of the present disclosure.

What is claimed is:

1. A method for preparing ethylene from chlorinated organic waste, comprising the steps of:
forming a plasma atmosphere from a working gas, and subjecting the chlorinated organic waste to pyrolysis reaction and staged quenching in sequence in the plasma atmosphere to obtain a pyrolysis product, wherein the staged quenching comprises a first quenching and a second quenching performed in sequence, a quenching rate of the first quenching being greater than a quenching rate of the second quenching; and the working gas contains hydrogen element and carbon element, a molar ratio of the hydrogen element to the carbon element in the plasma atmosphere being greater than 2:1; and
subjecting the pyrolysis product to gas-solid separation to obtain a gas-phase product and a solid-phase product, wherein the gas-phase product comprises ethylene.

2. The method as claimed in claim 1, wherein the quenching rate of the first quenching is greater than or equal to 1×10⁴ K/s, and a product obtained after the first quenching has a temperature of 1100-1300 K.

3. The method as claimed in claim 2, wherein the quenching rate of the second quenching is in a range of 300-1000 K/s, and the pyrolysis product has a temperature of less than 600 K.

4. The method as claimed in claim 1, wherein the working gas comprises at least one selected from the group consisting of dry gas, coke oven gas, and natural gas.

5. The method as claimed in claim 4, wherein the dry gas comprises at least one selected from the group consisting of reforming dry gas, hydrocracking dry gas, catalytic cracking dry gas, and coking dry gas.

6. The method as claimed in claim 1, wherein the chlorinated organic waste contains carbon element and chlorine element.

7. The method as claimed in claim 6, wherein the chlorinated organic waste comprises solid hexachlorobenzene waste or liquid tetrachloroethylene waste.

8. The method as claimed in claim 1, wherein the pyrolysis reaction is carried out at a temperature of 1800-3500 K.

9. The method as claimed in claim 1, wherein the staged quenching is carried out by means comprising inter-wall quenching and/or direct quenching;
the direct quenching comprises physical quenching and/or chemical quenching;
a cooling medium used in the inter-wall quenching comprises one selected from the group consisting of water, liquid nitrogen, a cooling oil, and a coolant; and
a cooling medium used in the direct quenching comprises at least one selected from the group consisting of argon, nitrogen, propane, and the working gas.

10. The method as claimed in claim 1, wherein the pyrolysis reaction is carried out in a plasma reactor; and
the plasma reactor comprises one selected from the group consisting of a radio frequency plasma reactor, a microwave plasma reactor, and an arc plasma reactor.

11. The method as claimed in claim 10, wherein the arc plasma reactor is a direct-current arc plasma reactor; and
the direct-current arc plasma reactor is a magnetically rotating arc thermal plasma reactor.

12. The method as claimed in claim 10, wherein under a condition that the chlorinated organic waste is solid hexachlorobenzene waste, the chlorinated organic waste is introduced into the plasma reactor via a solid feed system; and
under a condition that the chlorinated organic waste is liquid tetrachloroethylene waste, the chlorinated organic waste is pumped into the plasma reactor using a peristaltic pump.

13. The method as claimed in claim 1, wherein a flow rate of the working gas is in a range of 0.5-1500 Nm³/h; and
the chlorinated organic waste is treated in an amount of 0.5-1000 kg/h.

14. The method as claimed in claim 10, wherein the chlorinated organic waste is introduced into the plasma reactor using a carrier gas, and the carrier gas comprises at least one selected from the group consisting of dry gas, coke oven gas, hydrogen, and argon.

15. The method as claimed in claim 1, wherein the solid-phase product is elemental carbon.

16. The method as claimed in claim 1, wherein a conversion rate of the chlorinated organic waste is greater than 98%, and a yield of ethylene in the gas-phase product is greater than 40%.

17. The method as claimed in claim 2, wherein the staged quenching is carried out by means comprising inter-wall quenching and/or direct quenching;
the direct quenching comprises physical quenching and/or chemical quenching;
a cooling medium used in the inter-wall quenching comprises one selected from the group consisting of water, liquid nitrogen, a cooling oil, and a coolant; and
a cooling medium used in the direct quenching comprises at least one selected from the group consisting of argon, nitrogen, propane, and the working gas.

18. The method as claimed in claim 3, wherein the staged quenching is carried out by means comprising inter-wall quenching and/or direct quenching;
the direct quenching comprises physical quenching and/or chemical quenching;
a cooling medium used in the inter-wall quenching comprises one selected from the group consisting of water, liquid nitrogen, a cooling oil, and a coolant; and
a cooling medium used in the direct quenching comprises at least one selected from the group consisting of argon, nitrogen, propane, and the working gas.

19. The method as claimed in claim 7, wherein the pyrolysis reaction is carried out in a plasma reactor; and
the plasma reactor comprises one selected from the group consisting of a radio frequency plasma reactor, a microwave plasma reactor, and an arc plasma reactor.

20. The method as claimed in claim 11, wherein a flow rate of the working gas is in a range of 0.5-1500 Nm³/h; and
the chlorinated organic waste is treated in an amount of 0.5-1000 kg/h.
